(19) United States
(12) Patent Application Publication
(10) Pub. No.: US 2011/0118379 A1
(43) Pub. Date: May 19, 2011

(54) INTERVERTEBRAL DISC AND INTRAOCULAR LENS

(76) Inventors: Brian J. Tighe, Birmingham (GB); Valerie Franklin, Birmingham (GB); Fiona Jane Lydon, Birmingham (GB); Sally Roberts, Powys (GB); Jocelyn P.G. Urban, Oxford (GB); Sarit Sivan, Zichron Yaakov (IL)

(21) Appl. No.: 12/736,540

(22) PCT Filed: Apr. 20, 2009
(86) PCT No.: PCT/GB2009/001006

§ 371 Date: Feb. 1, 2011

(30) Foreign Application Priority Data
Apr. 18, 2008 (GB) 0807154.0

(51) Int. Cl. A61L 27/50; A61M 5/00

(52) U.S. Cl. 523/113; 604/191

(57) ABSTRACT

The invention relates to the use of an aqueous gel in the repair of or prevention of damage to soft tissue, the gel comprising an aqueous gel obtainable by polymerizing one or more olefinically unsaturated monomers comprising one or more phosphate, phosphonate, borate, sulphate and/or sulphonate groups. This is preferably used for intervertebral discs or a lens of an eye. Kits and syringes containing the components are also provided.

### BACKGROUND TO THE INVENTION

The invention relates to a material for use in repair of an intervertebral disc and a method for treating degeneration or repair of an intervertebral disc. The invention also relates to the use of the material as an intraocular lens.

The spinal column is formed from a number of vertebrae, which in their normal state are separated from each other by cartilaginous intervertebral discs. Each intervertebral disc acts to provide flexibility to the spinal column and to provide a mechanism for force distribution between the vertebrae. In the presence of a damaged disc, or in the absence of a disc, collapse of the intervertebral space occurs resulting in abnormal joint mechanics and premature development of arthritic changes in the spine.

A normal intervertebral disc has an outer ligamentous ring called the annulus fibrosus surrounding an inner nucleus pulposus. The annulus binds the adjacent vertebrae together and is constituted mainly of collagen fibres that are attached to the vertebrae and cross each other so that as the vertebrae are rotated in either direction half of the individual fibres tighten, thus resisting twisting or torsional motion. The nucleus pulposus is constituted of loose tissue, having about 85% water content. The nucleus hydration is maintained by the osmotic pressure arising from its high concentration of negatively charged aggrecan macromolecules.

The disc is always under load arising from body weight and muscle and ligament tensions. The hydrostatic pressure (which arises in the nucleus on account of its high water content) supports load, applies even stresses to the annulus and maintains disc height. The load on the disc is lowest when lying down and increases considerably when standing, bending, sitting or carrying weights and thus alters with every change of posture. As the disc is an osmotic system, fluid is expressed when pressure increases in the face of increased load on the disc and is re-imbibed when the load is reduced.

In disc degeneration, the nucleus pulposus loses aggrecan and consequently dehydrates and the disc loses height and cracks and fissures form within. Eventually the disc no longer acts hydrostatically, stress distributions across the disc and vertebral bodies are altered. Possibly as a result of nucleus pressure loss, the annulus also tends to desiccate, and becomes more rigid and becomes susceptible to fracturing or fissuring and the disc matrix may bulge or extrude out through the annulus wall (herniated disc). The extruded material can impinge on the spinal nerve root as it exits through the intervertebral disc foramen causing pain down the leg (sciatica). Disc herniation is readily diagnosed and surgical repair, if required, is usually successful. Herniation however only accounts for around 25% of back problems. Most back problems are associated with degeneration of the nucleus and lead to non-specific back pain.

Current treatment of non-specific back pain is either conservative or else involves invasive surgical procedures. Fusion is the main procedure and works on the principle of the removal of the degenerative intervertebral disc from between adjacent vertebrae followed by connecting the adjacent vertebrae with devices such as interbody cages which fuse two adjacent vertebrae together or Pedicle Screws which immobilise adjacent vertebrae relative to each other. Fusion however alters spinal biomechanics and is thought to lead to premature degeneration of adjacent discs. Hence disc replacements which attempt to recapitulate the biomechanical behaviour of healthy discs have been developed; these are either whole disc prostheses such as the Charité Artificial Disc (DePuy Spine, Inc., Raynham, Mass., USA) or the Acroflex disc (DePuy Spine, Inc., an elastic disc between titanium endplates), or nucleus replacements such as the Newcleus (Zimmer Spine, Minneapolis, Minn.) or PDN® (Raymedica, Inc., Bloomington, Minn.) involving insertion of a hydraulic disc that absorbs water and therefore swells following insertion of a partially dehydrated disc into the intervertebral space.

Existing materials and methods for repairing disc degeneration have the disadvantage that their use requires invasive surgery which is time consuming for surgeons, and therefore expensive, and is also painful for patients. In addition, some patients are not well enough for major surgery.

What is required is a material and method for repair of degenerate disc which is safe, can restore disc function and disc height, can prevent further load-related degenerative changes and development of spinal stenosis, is relatively non-invasive and does not require major surgery.

### BRIEF SUMMARY OF THE INVENTION

The present invention is directed to a material and to a method for repairing damage in, and/or preventing damage to, intervertebral discs. It is concerned more particularly with a material and a method for repairing and/or preventing damage to the portion of an intervertebral disc that has been subject to degeneration associated with back pain and/or to damage, such as herniation, as well as to a material and method for repairing that portion of an intervertebral disc remaining after the performance of a partial discectomy intervention. The invention may be particularly suitable for repairing or preventing damage to the discs of patients who are unfit for major surgery since the materials are easy to deliver to the patient in a low risk manner. Thus the invention may be useful for decreasing the incidence of spinal stenosis in, for example, elderly people who may otherwise be unfit for spinal surgery.

The invention also provides a method of replacing or augmenting soft tissue in other body sites. A particular area of application is the eye where soft tissue replacements are used, for example, in the form of intraocular lenses. It is usual for cataract operations to involve the implantation of an artificial lens following cataract removal. The various embodiments of the injectable soft tissue analogue, i.e. proteoglycan analogue or glycosaminoglycan analogue, disclosed herein provide the basis for a device such as an accommodating lens system in which the mechanical responsiveness of the gel is so designed to allow alteration of the refractive power of the eye in response to changes in tension of the ciliary muscle, thereby allowing the lens system to bring into focus on the retina images of objects that are both near and far from the eye.

A first aspect of the invention relates to the use of an aqueous gel in the repair of or prevention of damage to soft tissue such as the soft tissue of an intervertebral disc, the aqueous gel comprising: a gel obtainable by polymerizing one or more olefinically unsaturated monomers comprising one or more phosphate, phosphonate, borate, sulphate and/or sulphonate groups. Preferably, the monomers comprise one or more sulphate and/or sulphonate groups. The aqueous gel may be a water-swellable gel. The aqueous gel may be a water-swollen gel.

The polymer of the aqueous gel may be essentially an analogue of natural proteoglycan/glycosaminoglycan. By acting as an analogue of the natural proteoglycan/glycosaminoglycan, the polymer is able to respond to osmotic forces and thereby aid in the restoration of disc height.

It is preferred that polymerization of the monomers is carried out in the presence of a cross-linking agent. Gelled polymer is more easily contained within the disc than a non-gelled polymer, therefore increasing the speed of polymerization, i.e. gelation is likely to be beneficial. This has the advantage of the patient being able to recover more quickly.

It is also preferred that the monomer has one of the following formulae:

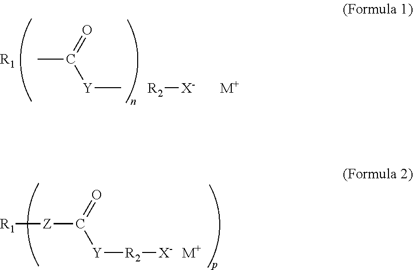

in which:
R₁=olefinically unsaturated moiety,
R₂=branched or unbranched, saturated or unsaturated, substituted or non-substituted alkyl, or substituted or unsubstituted aryl,
X=phosphate, phosphonate, borate, sulphate or sulphonate group,
Y=—NH— or O,
Z=independently a single bond or —CH₂—,
M=a cation,
n=0 or 1, and
p=1 or 2.

Preferably R₁ is an ethenyl or propenyl moiety.

An ethenyl group is, for example:
—CH═CH₂

A propenyl group is, for example:
—CH═CH—CH₃ or —CH₂—CH═CH₂

Preferably, R₂ contains from two to six carbon atoms, for example, two, three, four, five or six carbon atoms. More preferably, R₂ contains three or four carbon atoms. Most preferably, R₂ is selected from propyl, secondary butyl or tertiary butyl.

Preferably, X is sulphate or a sulphonate.

Preferably the sulphonate is a C₁, C₂, C₃, C₄, C₅, C₆, C₇, C₈, C₉, C₁₀, C₁₁, C₁₂, C₁₃, C₁₄, C₁₅, C₁₆, C₁₇, or C₁₈ sulphonate.

Preferably the cation is selected from metal cations, ammonium cation and a proton.

It is preferred that the aqueous gel has a fixed charge density. More preferably the aqueous gel has a fixed negative charge density. An advantage of a fixed charge density is that it enables the polymer to maintain and regulate its hydration and therefore maintain and regulate the hydration of the disc into which it has been delivered and thereby maintain and regulate disc height.

Preferred fixed charge densities include from about 4.1 to about 4.4 moles/kg, particularly from about 4.1 to about 4.3 moles/kg and from about 4.2 to about 4.4 moles/kg, especially about 4.3 (e.g. 4.29) moles/kg or about 4.2 (e.g. 4.18) moles/kg.

It is also preferred that the monomers are anionic.

Preferably, the monomers are selected from: sodium-2-(acrylamido)-2-methylpropane sulphonate (sodium AMPS), ammonium-2-(acrylamido)-2-methylpropane sulphonate (ammonium AMPS), 3-sulphopropyl (meth)acrylate potassium salt, 3-sulphopropyl (meth)acrylate sodium salt (SPA-sodium salt), di-potassium salt of bis-3-sulphopropylester itaconate, di-sodium salt of bis-3-sulphopropylester itaconate, styrene sulphonate, vinyl sulphonate polyethylene glycol acrylate and combinations, e.g. mixtures, thereof. More preferably, the monomers are selected from: sodium-2-(acrylamido)-2-methylpropane sulphonate, 3-sulphopropyl (meth)acrylate potassium salt, and 3-sulphopropylester acrylate.

It is preferred that polymerization is carried out in the presence of a co-monomer. Any suitable co-monomer may be used. For example, the co-monomer may be selected from: one or more of an acrylic, methacrylic, acrylamido or vinyl compound capable of undergoing free radical polymerization, esters of unsaturated polyhydric alcohols (e.g. butenediol), vinyl cyclic compounds (e.g. styrene, vinyl furane, N-vinyl pyrrolidone), unsaturated acids (e.g. acrylic, methacrylic, propacrylic acid), unsaturated anhydrides (e.g. maleic, citraconic, itaconic), unsaturated nitriles (e.g. acrylonitrile, methacrylonitrile), unsaturated amines (e.g. acrylamide, dimethylaminoethyl methacrylate), vinyl halides (e.g. vinyl chloride, vinyl iodide, allyly chloride), unsaturated ketones (e.g. methyl vinyl ketone, ethyl vinyl ketone), unsaturated ethers (e.g. methyl vinyl ether, diallyl ether), unsaturated esters (e.g. hydroxylethyl methacrylate, hydroxypropyl acrylate), and unsaturated functional silanes, alkyl methacrylates (e.g. methyl methacrylate, ethyl methacrylate) and combinations, e.g. mixtures, thereof.

Preferably the compound capable of undergoing free radical polymerization is selected from: 2-hydroxyethyl methacrylate (HEMA), glycerol methacrylate, polyethyleneglycol mono(meth)acrylate, methacrylic acid and acrylic acid, N,N-dimethyl acrylamide (DMA), 2-hydroxyethyl methacrylamide, 2-hydroxyethyl acrylamide, 2,2-di methoxy, 1-hydroxy acrylamide, hydroxymethyl diacetone acrylamide, N-acryloyl morpholine, N-(tris(hydroxymethyl)methyl)acrylamide, 2-hydroxylmethylacrylamide, N-vinyl lactams such as N-vinylpyrrolidone (or NVP), N-vinyl amides such as N-vinyl acetamide, N-vinyl-N-methyl acetamide, N-vinyl-N-ethyl acetamide, N-vinyl-N-ethyl formamide, N-vinyl formamide, and combinations, e.g. mixtures, thereof.

The monomers and/or co-polymers may comprise one or more of the following physiologically compatible cations: Na⁺, Ca⁺, K⁺, NH₄⁺, and combinations, e.g. mixtures, thereof. The cations may be ionically associated with the monomer and/or co-monomer. Preferably the cations are anionically bound to the monomer and/or co-monomer. Particularly preferred cations include Na⁺ and K⁺.

It is preferred that the aqueous gel has a ratio of Na⁺ to K⁺ approximately the same as the ratio of Na⁺ to K⁺ present in vivo, for example in a human or an animal, in particular in an intervertebral disc nucleus pulposus or in the lens of an eye of a human or an animal. Use of both AMPS and SPA allow gel formulations to be optimised to produce a ratio that gives a good clinical outcome.

Preferred cross-linking agents include methylene bisacrylamide, ethylene glycol dimethacrylate, polyethylglycol di(meth)acrylate, tetraethylene glycol dimethacrylate, diallyl tartramide, pentaerithratol diacrylate, divinyl benzene, polyethylene glycol diacrylate (IRR-280), a multifunctional macromer (such as Nelfilcon), acrylamido sulphonic acid and acrylamido sulphonate, 2-acrylamido-2-methylpropane-sulphonic acid, any water soluble oligomer or polymer comprising at least two polymerizable double bonds and combinations, e.g. mixtures thereof.

Macromers may be synthesised by reacting water soluble hydroxyl-containing molecules with N-methylol acrylamide.

Typically, macromers suitable for this application are polymers comprising 4 or more monomeric subunits and have 2 or more functional groups.

The synthesis of cyclodextrin acrylamidomethyl (CD-NMA) has been described by Lee, M. H. et al (Journal of Applied Polymer Science, 2001, Vol 80, pages 438-446).

The synthesis of acrylamidomethyl chito-oligosaccharide (COS-NMA) has been reported by Song, J. W. et al (Journal of Polymer of Science: Part A: Polymer Chemistry, 2001, Vol 39, pages 1810-1816).

The synthesis of acrylamidomethyl cellulose acetate has been described by Kumar, R. N. et al (Carbohydrate Polymers, 2006, Vol 64, pages 112-126.)

A polyvinyl alcohol-based macromer may be prepared by dissolving 10 grams of PVA solution (molecular weight 30,000 Da) in 100 grams of water and adding 0.1 g acrylamidomethyl (NMA) and 1 g of 20% hydrochloric acid solution to the reaction medium. The reaction vessel is then placed in a water bath at 60° C. for 4 h.

It is not necessary to add additional inhibitor to prevent premature polymerization of the acrylamido double bonds if the NMA already contains an inhibitor such as MEHQ (4-methyl ethyl hydroquinone) inhibitor. The reaction may be stopped by placing the reaction vessel in a bath of ice-cold water. A small amount of sample may be then placed in a NMR tube to calculate the extent of reaction.

Although the cross-link density is an important factor in controlling the swelling behaviour of sulphonate gels, its primary function is to ensure that the protecglycan/glycosominoglycan analogue behaves as an elastic gel and not as a viscous fluid. Inclusion of a very low level of cross-linker is able to produce an elastic gel.

It is further preferred that the water-soluble polymer comprises a water-soluble interpenetrant, for example acrylamide, polyacrylamide and/or a substituted vinyl amide, polymerized substituted vinyl amide and combinations, e.g. mixtures, thereof.

The time taken for polymerization of the monomers to form a polymeric gel may be adjusted by varying the nature and concentration of cross-linking agents and/or initiators.

An initiator may be used to increase the rate at which the monomers polymerize in situ.

The initiator may be a two-part initiator, i.e. an initiator in which each of the two parts of the initiator is individually stable in contact with the monomers but when combined in the presence of the monomers and/or a liquid they bring about polymerization.

The two parts of the two-part initiator may be conveniently stored in separate portions, one or more of the portions may also contain monomers. When the separate portions are brought together, polymerization is initiated.

The concentrations of the two parts of the initiator and/or the temperature and/or the content and/or relative volumes of the portions may be adjusted to achieve polymerization over a suitable time frame.

By bringing the two portions together immediately before introducing them into the disc cavity, the time taken for the system to achieve polymerization is predictable. A further element of control is achieved by the fact that the interaction of the two parts of the initiator, and thus the polymerization time, is temperature-dependent. If the portions are mixed at a temperature below normal body temperature (e.g. 20° C.), the system may be more effectively ‘triggered’ when the mixed solutions are delivered into the disc cavity since the disc cavity will be at around a temperature of 37° C.

The two-part initiator may comprise a reducing agent and an oxidising agent. As the initiator it is possible in principle to employ all initiators and redox initiator systems, comprising an oxidizing agent and a reducing agent, that are suitable for this purpose. Examples of suitable oxidizing agents are hydrogen peroxide and organic peroxides, such as benzoyl peroxide, t-butyl hydroperoxide, di(isopropyl) peroxydicarbonate, t-butyl peroctoate, bis(4-t-butylcyclohexyl) peroxydicarbonate, isopropyl peroctoate and other free radical organic peroxides, persulfates, such as sodium persulfate, potassium persulfate and the like. Examples of suitable reducing agents are hydroxymethanesulfinic acid and its salts, especially the sodium salt, ascorbic acid, sodium metabisulfite, acetone bisulfite, N,N-dimethyl toluidine and the like.

Particularly preferred initiators include hydrogen peroxide/ascorbic acid, t-butyl hydroperoxide/acetone bisulfite, N,N, N′,N′-tetramethyldiamine and t-butyl hydroperoxide/sodium hydroxymethanesulfinate. Particularly suitable oxidising agents include potassium persulphate and oxypersulphate. Ascorbic acid is a particularly suitable reducing agent.

Other suitable initiators include a free radical catalyst, a Ziegler type catalyst, irradiation, persulphates, microwave and/or thermal techniques.

The storage stability of peroxymonosulfate solutions can be increased by the generally known use of stabilisers such as dipicolinic acid (DPA), complexing agents—for example phosphonates such as hydroxyalkylidene diphosphonates and diethylene triamine penta(methylene phosphonic acid) [EDTPA]. The rate of polymerization is affected by the well known inhibitory effect of oxygen in addition to the presence of stabilizers. The particular choice of stabilizer system may influence the need for exclusion of oxygen by degassing the monomer mixture (for example by passing a stream of nitrogen gas through the monomer solution).

Preferably the solutions and components making the invention are packaged in oxygen—excluding packaging prior to use.

Preferably the polymer is capable of curing in situ but incapable of curing ex situ. This has the advantage that the polymer is in liquid form ex situ and therefore is easily manageable, for example easily managed within a delivery apparatus such as a syringe. Thus the components described act as precursors of the resultant polymer which forms on polymerization of the precursors.

It is preferred that the aqueous gel comprises a marker suitable for in vivo use, e.g. a radiopaque marker. Suitable radiopaque markers include, for example iohexyl (e.g. Omnipaque) and iomeprol (e.g. Iomeron 300).

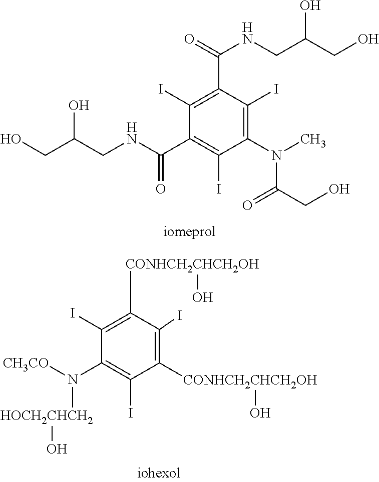

The monomers may be delivered to a target site with a multiple-barrel syringe, a first barrel of the syringe containing a first part of the two-part initiator and a second barrel of the syringe containing a second part of the two part initiator such that on expulsion from the syringe, the monomers and the first and second parts of the two-part initiator come into contact causing the monomers to polymerize at the target site.

A multiple barrelled syringe may deliver fluid from the barrels to a needle through a mixing chamber. The mixing chamber may be plain, i.e. empty. Alternatively the mixing chamber may comprise a baffle, for example a helical length of rigid or semi rigid material such as a plastic, this may be referred to as a ‘corkscrew’. The baffle may be as long as the length of the chamber or shorter than the length of the chamber, for example 50, 60, 70, 80, 90 or 100% of the length of the chamber. The baffle may vary in the number of turns it comprises. Preferred baffles comprise 1, 2, 3, 4, 5, 6, 7, 8, 9, 10, 11, 12, 13, 14, 15 or 16 turns. The skilled person can select a baffle with an appropriate number of turns based upon the speed at which the contents of the barrel is to be expelled and the reactivity of the solutions being expelled. The reactivity of the solutions is dependent upon, for example, the amount of redox components present.

The presence of a baffle is optional, adequate mixing can be achieved without a baffle. Furthermore, baffles which are small and/or have a minimal number of turns also provide excellent mixing and produce minimal resistance to expulsion of the fluid from the barrel.

Preferably the marker and one part of the two part initiator is packaged as one component of the polymer precursor and the monomers, cross-linking agent and a second part of the two part initiator is packaged as a second component of the polymer precursor.

Preferably the syringe is a dual barrelled syringe. Preferably the syringe comprises an 8 turn mixer. Preferably the syringe comprises a blunt ended mixing cavity. Preferably the syringe is attached to a 19 gauge needle. The length and diameter of the needle used can be selected according to the requirements of a particular clinical situation.

The target site may be the intervertebral disc, particularly areas of the disc such as degenerate nucleus pulposus. The target site may be in a human or an animal, for example domestic or agricultural animals such as dogs, cats and horses.

It is preferred that the monomers are provided in a concentrated solution. Preferably, the solution does not comprise a solvent which damages cell membranes. More preferably, the solution is an aqueous solution. Preferably the monomers are delivered to a target site by injection.

The polymer may be fully or partially hydrated. A partially hydrated polymer may become fully hydrated following delivery to a target site. Hydration may be achieved through absorption of a liquid, e.g. water, from, e.g., surrounding body tissue. Thus the polymer mimics the ability of natural proteoglycan to rehydrate diurnally i.e. the process by which loss in disc height is brought about during the day and is regained at night.

Preferably, the components of the aqueous gel are sterilized prior to use. Sterilization may be carried out by any suitable method including those used in the manufacture of pharmaceutical preparations such as filtration.

A second aspect of the invention relates to the use of a an aqueous gel, as described for the first aspect of the invention, in the replacement or augmentation of soft tissue in the eye. For example, the soft tissue may comprise the lens. The gel may be used as an intraocular lens, for example an artificial lens. The artificial lens may be useful following cataract removal.

The aqueous gel may be used in an accommodating lens system. For example, the mechanical responsiveness of the gel may be so designed to allow alteration of the refractive power of the eye in response to changes in tension of the ciliary muscle, thereby allowing the lens system to bring into focus on the retina images of objects that are both near and far from the eye.

A third aspect of the invention provides a proteoglycan replacement material comprising an aqueous gel obtainable by polymerizing one or more olefinically unsaturated monomers comprising one or more phosphate, phosphonate, borate, sulphate and/or sulphonate groups and a marker in which the marker is suitable for in vivo use. The aqueous gel is preferably the same as that described in relation to that of the first aspect of the invention.

A fourth aspect of the invention provides a kit comprising an aqueous gel for the repair of or prevention of damage to an intervertebral disc and/or for the replacement or augmentation of soft tissue in the eye, the aqueous gel comprising: an aqueous gel obtainable by polymerizing one or more olefinically unsaturated monomers comprising one or more phosphate, phosphonate, borate, sulphate and/or sulphonate groups. The aqueous gel is preferably the same as that described in relation to that of the first aspect of the invention. The soft tissue may be the lens. For example, the aqueous gel may be used as an intraocular lens.

A fifth aspect of the invention provides a multiple barrel syringe containing one or more olefinically unsaturated monomers comprising one or more phosphate, phosphonate, borate, sulphate and/or sulphonate groups in which:
a. a first barrel of the syringe contains a first part of a two-part initiator and
b. a second barrel of the syringe contains a second part of a two-part initiator
   such that when the contents of the syringe are released from the syringe, the monomers, the first part of the two-part initiator and the second part of the two-part initiator contact each other causing polymerization of the monomers to form an aqueous polymeric gel.

Preferably the multiple barrel syringe is a two-barrel syringe.

Preferably the aqueous polymeric gel is as described for the first aspect of the invention.

It is preferred that the syringe and the components of the aqueous gel are sterilized prior to use. Suitable sterilization methods include those described in relation to the first aspect of the invention.

A sixth aspect of the invention provides a method of repairing and/or preventing damage to an intervertebral disc comprising delivering an aqueous gel obtainable by polymerizing one or more olefinically unsaturated monomers comprising one or more phosphate, phosphonate, borate, sulphate and/or sulphonate groups to the intervertebral disc.

Preferably, concentrated solutions of the precursors of the polymer, i.e. the monomers and optionally other components described above, are delivered into the space normally occupied by the nucleus pulposus. Preferably, the precursors are induced in situ to polymerize to form a polymeric gel. Preferably, the solution does not comprise a solvent which damages cell membranes. More preferably, the solution is an aqueous solution. Preferably the precursors are delivered to the target site by injection.

Preferably the aqueous polymeric gel is as described for the first aspect of the invention.

The aqueous polymer may be delivered in a fully or partially hydrated state. A partially hydrated polymer would become fully hydrated in situ at the target site by absorbing liquid, e.g. water, from surrounding body tissue. Thus the polymer mimics the ability of natural proteoglycan to rehydrate diurnally i.e. the process by which loss in disc height is brought about during the day and is regained at night.

A seventh aspect of the invention provides a method of replacing or augmenting soft tissue in the eye comprising delivering an aqueous gel obtainable by polymerizing one or more olefinically unsaturated monomers comprising one or more phosphate, phosphonate, borate, sulphate and/or sulphonate groups to the eye, for example to the lens.

Preferably the aqueous polymeric gel is as described for the first aspect of the invention.

The aqueous polymer may be delivered in a fully or partially hydrated state. A partially hydrated polymer would become fully hydrated in situ at the target site by absorbing liquid, e.g. water, from surrounding body tissue. Thus the polymer mimics the ability of the natural lens to rehydrate.

An eighth aspect of the invention provides a replacement intervertebral disc comprising an aqueous polymer as described in relation to the first aspect of the invention. The replacement disc may comprise a rigid or semi-rigid chamber for enclosing the aqueous polymer. The chamber may comprise a metal and/or a ceramic and/or other substance.

A ninth aspect of the invention provides an ocular device such as an intraocular lens comprising an aqueous polymer as described in relation to the first aspect of the invention. The ocular device may comprise a rigid or semi-rigid chamber for enclosing the aqueous polymer.

For all aspects of the invention, preferably the soft tissue, e.g. intervertebral disc or lens, is a human or animal soft tissue, preferably a human soft tissue.

### BRIEF DESCRIPTION OF THE FIGURES

The invention will be described, by way of example only, with reference to the following figures:

FIG. 1 is a schematic graph showing the relationship between volume swell and ionic strength for an AMPS-based proteoglycan analogue. (A) High Dilution: charge repulsion and osmotic effects balanced by retractive force of cross-links, (B) increasing salt concentrations screens negative charge repulsion, osmotic effects are more significant, (C) salt screens charge repulsion, hydrophilicity of monomers predominates.

FIG. 2 is a graph showing the relationship between swelling pressure and fixed charge density (FCD) of experimental proteoglycan analogues. ♦ 14 (178 FCD/gr dry wt), □ 13 (073 FCD/gr dry wt), ▴ 18 (132 FCD/gr dry wt), ▪ 16 (076 FCD/gr dry wt), − Nucleus (0.8-1.4 FCD/gr dry wt).

FIG. 3 is graphs showing MTS cytotoxicity assay results at 24 hours of (A) L929 cells cultured in ACMO and (B) bovine nucleus pulposus cells cultured with sodium AMPS. Results are a mean of three assays. FIG. 3 shows the percentage of viable cells relative to values obtained over the same period with a positive non-toxic tissue culture plastic control material.

FIG. 4 is photographs of typical cross-sections of motion segments after incubating with trypsin showing (A) the cavity injected with radiopaque dye, and (B) the injected proteoglycan analogue labelled with red dye or (C) micrograph of section stained with haematoxylin and eosin. The scale shows large graduations of 10 mm and small graduations of 1 mm.

FIG. 5 is photographs of the typical appearance of discs injected with polymerized construct following exposure to (A) no load and (B) two hours static loading at 0.2 MPa, 37° C. A similar appearance was apparent after cyclic loading at walking speeds (not shown). The scale shows large graduations of 10 mm and small graduations of 1 mm.

### DESCRIPTION OF PREFERRED EMBODIMENTS

Polymers were prepared according to Table 1 and Table 2. The principal variations were: fixed charge density (FCD) of the sulphonate (obtained using different ratios of carboxyl, amide and methoxy PEG substituted monomers as diluents), density (using conventional difunctional cross-linking agents of differing molecular weights and multi-functional macromer cross-linking agents) and the presence and nature of water soluble interpenetrants (such as acrylamide and substituted vinyl amides).

A series of multi-functional macromers based both on polyvinyl alcohol and on a series of AMPS/SPA copolymers with neutral monomers, by reaction of pendant hydroxyl groups with N-methylol acrylamide were used. These behaved both as reinforcing interpenetrants and multi-functional cross-linking agents.

Proteglycan analogue-forming polymerizations were carried out with a wide range of water-soluble two-pack redox initiation systems since an injectable surgical product would benefit from this. The ascorbic acid-oxypersulphate (Oxone) pair has emerged as the preferred redox combination.

TABLE 1

| | | | PEG | Multifunctional | PEG400 | | Total | Sulphonate | | Charge |
|---|---|---|---|---|---|---|---|---|---|---|
| | NaAmps | KSPA | Acrylate | macromer | diacrylate | Water | Dry Wt | Weight | Sulphonate | density |
| | (g) | (g) | (g) | (g) | (g) | (g) | (g) | (g) | (%) | (moles/kg) |
| SD 2 | 1.44 | — | 0.62 | 0.04 | — | 4.0 | 2.1 | 0.5026 | 23.9 | 2.99 |
| SD 4 | 1.21 | — | 1.12 | 0.04 | — | 3.9 | 2.37 | 0.4290 | 18.1 | 2.26 |
| 13 A | 1.74 | 1.4 | — | — | 0.2 | 5.6 | 3.34 | 1.0900 | 32.6 | 4.08 |
| 13 | 1.74 | 1.4 | — | 0.12 | — | 5.8 | 3.26 | 1.0900 | 34.3 | 4.29 |
| 18 | 1.74 | 1.4 | — | 0.8 | — | 4.8 | 3.94 | 1.0900 | 32.2 | 4.03 |
| 20 | 3.48 | — | — | — | 0.2 | 3.0 | 3.68 | 1.2142 | 33.4 | 4.18 |

TABLE 2

| | | | PEG | Multifunctional | PEG 400 | | Total | Sulphonate | | Charge |
|---|---|---|---|---|---|---|---|---|---|---|
| | NaAmps | KSPA | acrylate | macromer | diacrylate | Water | Dry Wt | Weight | Sulphonate | Density |
| | (g) | (g) | (g) | (g) | (g) | (g) | (g) | (g) | (%) | (moles/kg) |
| 16 | 1.74 | 1.4 | — | 0.8 | — | 5.3 | 3.94 | 1.0900 | 32.2 | 4.03 |
| 18 | 1.74 | 1.4 | — | 0.8 | — | 4.8 | 3.94 | 1.0900 | 32.2 | 4.03 |
| 14 | 1.74 | 1.4 | — | 0.2 | — | 4.1 | 3.94 | 1.0900 | 34.0 | 4.25 |

KSPA (potassium salt of 2-sulphopropyl acrylate) has the following formula:

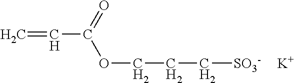

NaAMPS (2-acrylamido 2,2 methylpropane sulphonic acid) has the following formula:

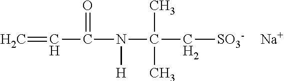

PEG acrylate (polyethylene glycol acrylate) is a neutral monomer.

PEG diacrylate (Polyethylene glycol 400 diacrylate) is a difunctional cross-linking agent.

Multifunctional macromer is a multifunctional cross-linking macromer based on polyvinyl alcohol prepared by the well-known reaction of pendant hydroxyl groups with N-methylol acrylamide described above.

The level of sulphonate and the charge density were determined for each of the compositions.

The compositions of Table 1 showed good swelling behaviour (i.e. no anomalous data points at particular concentrations). The compositions shows some variations in some of fixed sulphonate charge density, cross-linking agent, nature and concentration of other (e.g. neutral) monomers, dilution of the sample (i.e. water content as polymerized).

The swelling behaviour of compositions 13 and 18 was similar despite their different charge densities. This could be due to the effect of dilution on the conformation of the network on formation.

Table 2 compares three compositions with identical charge density but polymerized with different levels of water. Swelling increases with decreasing levels of dilution.

In the compositions of Tables 1 and 2, (a) PEG 400 diacrylate was a less efficient cross-linker than multifunctional macromer, and (b) the levels of multifunctional macromer have been chosen to lie in a range where variations have more effect on strength of the isolated gel produce than on its swelling behaviour.

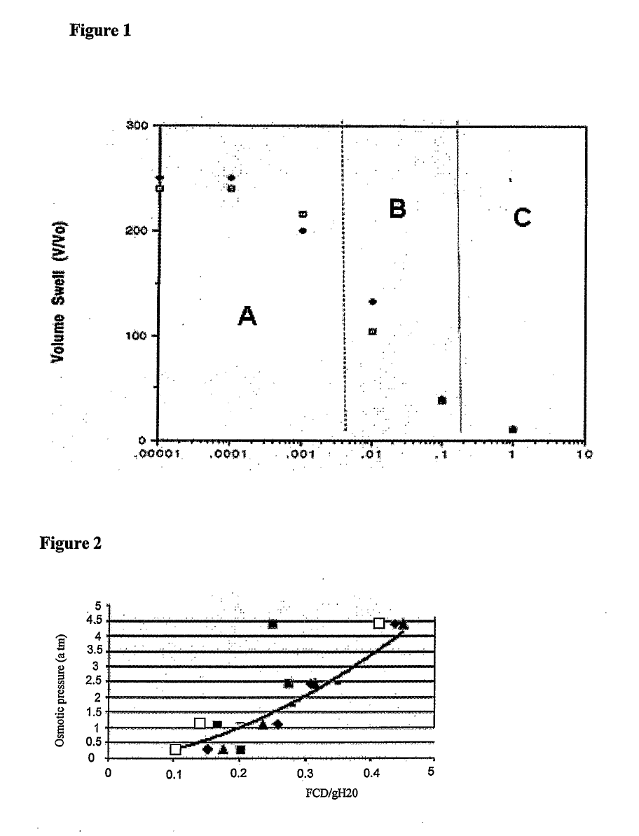

The effect of osmolarity on swelling behaviour of the structural variants was examined in order to determine the preferred ways of achieving matches to the characteristic age-related swelling behaviour of natural disc. The generalised swelling behaviour of a copolymer with a fixed charge density of 3 moles/kg is shown in FIG. 1 with an indication of the changing importance of the factors that influence swell as osmolarity is increased.

A range of techniques was used to monitor both the progress of polymerization (including onset of gelation, development of mechanical properties, level of residual monomers as a function of all intrinsic and extrinsic polymerization variables) and the behaviour (physico-chemical and mechanical) of the formed analogue. Briefly, gels were prepared by injecting into Petri dishes, into small balloons and into bovine and porcine models of the human disc.

A series of analogues designed to mimic the swelling behaviour of natural disc tissue were thus prepared as described in Tables 1 and 2. The physico-chemical properties of these analogue formulations were compared with those of natural disc. FIG. 2 shows the fixed charge density of gels in relation to their swelling pressure and compares it to that of intervertebral disc.

FIG. 2 demonstrates that the experimental analogues show good approximation to a typical sample of disc nucleus but that calculated fixed charge density is not the only factor controlling swell behaviour of the synthetic analogues. Interestingly, increased dilution at the point of the polymerization of the network reduces the effective fixed charge density. From these data and data on rates of swelling and compression, composition 13 was chosen as a suitable example to demonstrate the applicability of the invention using in vitro studies.

This is further demonstrated by Table 3 which shows that the materials of Tables 1 and 2 behaved in a comparable manner to healthy human disc nucleus when an osmotic pressure was applied.

TABLE 3
Variation of the fixed charge density (FCD) of different
hydrogel compositions (on total water basis) with applied
osmotic pressure compared to that of a healthy human
disc nucleus (Nucleus) between 30-40 years.

| | | Material | | | | |
|---|---|---|---|---|---|---|
| | | Nucleus | 13 | 14 | 16 | 18 |
| Applied | 0.25 | 0.12 | 0.1 | 0.15 | 0.2 | 0.175 |
| osmotic | 1.1 | 0.2 | 0.14 | 0.26 | 0.16 | 0.24 |
| pressure | 1.75 | 0.28 | — | — | — | — |
| (atmospheres) | 2.4 | 0.35 | 0.315 | 0.31 | 0.28 | 0.32 |
| | 4.4 | 0.45 | 0.425 | 0.445 | 0.26 | 0.455 |

Materials 13, 14, 16 and 18 are as described in Tables 1 and 2.

Cytotoxicity testing was carried out on precursor monomers and proteoglycan analogues via the MTS assay using a commercial Kit (MTS assay Cell titre 96® AQᵤₑₒᵤₛ One Solution Cell Proliferation Assay). Briefly, 5×10³ cells per well were allowed to adhere to a 96 well plate for 24 hours. The substance under test was added and incubated with the cells for 24 hours before assessment of cell activity with MTS solution. Two cell types were used: (i) L929 mouse fibroblasts (European Collection of Cell Culture: ECACC No 85011425) which are typically used in cytotoxicity tests and (ii) bovine nucleus pulposus cells, prepared by enzymatic digestion of nucleus pulposus from bovine coccygeal discs.

The individual possible components of proteoglycan analogues were tested, including: NaAMPS, KSPA, acryloyl morpholine (ACMO), glycerol, ascorbic acid, Oxone (potassium oxypersulphate), PEG diacrylate and multifunctional macromer cross-linkers. The proteoglycan analogues are exemplified by those in Tables 1 and 2. The initiators, ascorbic acid and Oxone, and cross-linking agents were found not to be toxic to L929 or bovine disc cells tested.

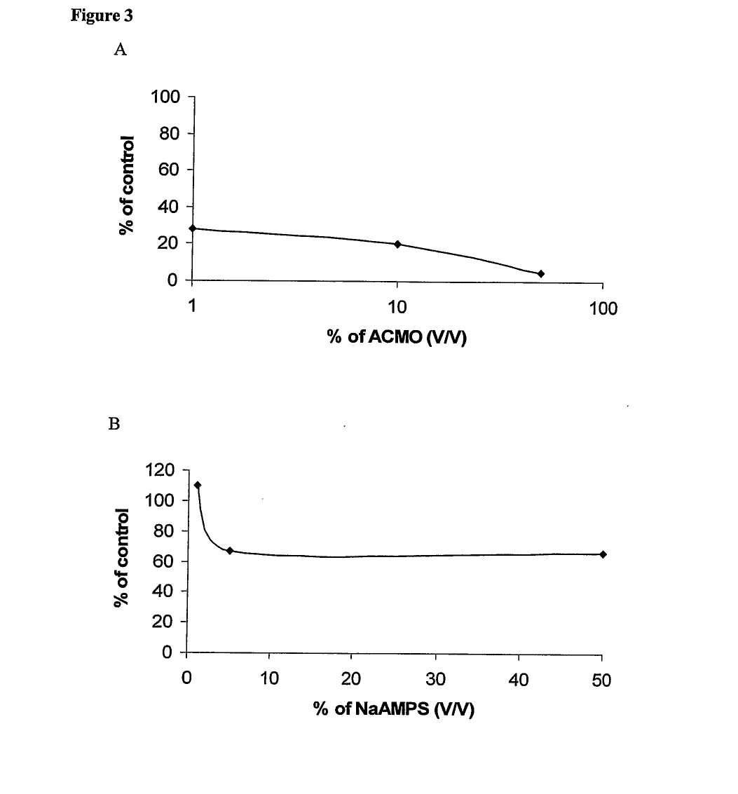

The toxicity at 24 hrs of ACMO and NaAMPS as a function of concentration are illustrated in FIGS. 3A and 3B.

The concentration dependence shown in FIG. 3B is typical of the effect seen with ionic monomers and is due to the effect of increasing concentration on the osmolarity of the culture medium. Since the monomer is converted within minutes to low toxicity polymer and residual monomer levels will only be of the order of 1%, the cytotoxicity results provide encouraging results for the concept of an injectable AMPS based proteoglycan analogue. This contrasts with the behaviour of conventional organic monomers such as ACMO and demonstrates that the use of this monomer as a charge diluent (which has been found to be unnecessary) would produce a much less desirable situation.

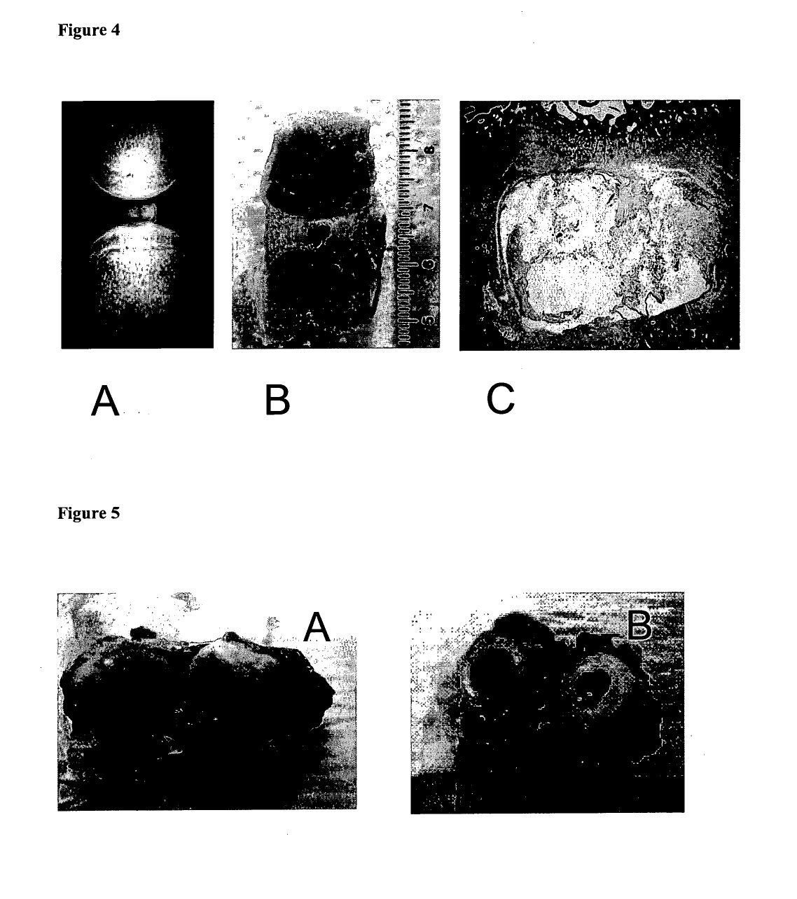

Bovine coccygeal motion segments were used to develop in house model system to mimic disc degeneration. This was then used for trial injections of the PG analogue into the disc and for subsequent testing. Motion segments were injected centrally with either trypsin (20 mg/ml) or papain (20 mg/ml) and cultured at 37° C. for up to 3 weeks. This produced disruption which could be visualised by injection of radio-opaque marker (FIG. 4A). By week 3 treated discs showed very low levels of proteoglycan content as measured by dimethylmethylene blue assay (DMB) compared to control discs. Pre-treated motion segments were used for injection of the PG analogue as a two-pack system using either a single syringe, mixing solutions just prior to using, or via a dual syringe system. For the dual syringes the mix was split into 2 portions, one containing the reducing agent ascorbic acid and the other containing the oxidising agent, oxone. Various syringes were tried and tested, the best system being a dual barrelled syringe, with an 8 turn mixer in a blunt ended mixing cavity attached to a 19 G needle. Explants of models of degenerate bovine discs injected with the PG analogue can be seen in FIG. 4 (B and C).

Model degenerate discs were produced as described above and injected under a load equivalent to that found in an L3-L4 disc in a prone posture (load data: Wilke H J, Neef P et al. Spine (1999) 24:755-62). Discs were injected with the composition 13 (see Table 1 for details), containing a red dye for easy visualization and the initiator in the cold and were then allowed to polymerize at 37° C. The preferred protocol and initiator composition is described below:

Compositions are split into two solutions to prevent polymerization taking place prior to injection. Typically one solution (solution A) contains:
monomer or monomers (NaAMPS is a 58% solution with water so solution A will contain some water from this solution)
cross linker e.g. water soluble multifunctional macromer, Nelfilcon, IRR 280 or ebacryl II
part 1 of a dual initiator system usually 0.1 M ascorbic acid.

The second solution (solution B) contains:
water (balanced to take into account the water contained in the NaAMPS solution)
part 2 the initiator system e.g. 0.1 M oxone or 0.1 M potassium persulphate.

Both solutions can be made in advance and stored in the refrigerator prior to use. Several syringes with volumes sizes from 4 ml to 14 ml were used together with a selection of mixing chambers with helical baffles ranging from 6 to 16 turns and lengths of 3 mm and 4.6 mm. All gave satisfactory results but the smaller syringe with the most effecting mixing chamber was chosen for further work in the laboratory. If the device were to be used in a surgical procedure the solution would be suitable to pass through a discogram needle (for example a 19 gauge needle) via the mixing chamber.

A characteristic composition is as follows:

Solution A: 3 g NaAMPS 58% solution (1.74 g NaAMPS and 1.26 g water)
1.4 g SPA
0.12 g water soluble multifunctional macromer such as Nelfilcon
0.65 g of 0.1 M ascorbic acid

Solution B 0.65 ml of 0.1M potassium persulphate
4.52 g water.

Once each component is placed into the dual syringe it can be stored in a refrigerator until required.

The injected discs were then loaded in two modes: (i) static compressive loading (0.2 MPa for 2 hrs at 37° C. equivalent to the resting load on lumbar discs) and (ii) cyclic loading (4 kg (equivalent to standing load), 600 cycles at a frequency of 1 Hz (walking frequency), at room temperature).

Discs were subsequently dissected and the presence of hydrogel noted (FIGS. 5A and 5B).

A strong indication of the success of the invention in acting as an injectable analogue of natural disc tissue was seen in the fact that the consistency of the gel in the dissected disc was similar to that of that of an adolescent disc, being highly hydrated and semi-fluid. No leakage of gel from the disc was apparent, the gel appeared to fill the degenerate cavity and remain stable throughout the test period. A similar appearance was apparent after cyclic loading at walking speeds (not shown). Appropriate loads were determined according to Wilke H J, Neef P et al. Spine (1999) 24:755-62.

The inventors have identified the parameters important to the development of an optimized injectable proteoglycan replacement material. For in situ efficacy, the level of hydration and hydration response of the proteoglycan analogue are the most important controlling factors. Mechanical properties, as measured on isolated unconstrained gel, are much less relevant when the gel is constrained in the disc space. The two factors that are important for a clinically useable product are (a) the robustness of the gelation time and (b) the incorporation of a radiopaque marker. Levels of inhibitor in the monomers, levels of oxygen in the monomers at the point of injection, temperature and storage conditions have an effect on the reproducibility of individual compositions, as would be expected for a free radical polymerization.

### Further Initiation System Example

| | KSPA | AMPS | | PEG | TEMED | KPS | Oxygen | Gelation |
|---|---|---|---|---|---|---|---|---|
| Component | (50%)/g | (50%)/g | H2O/G | acrylate/g | (0.4M)/g* | (0.18M)/G | Exclusion | Time (min) |
| Amount | 3 | 3 | 1.3 | 0.05 | 0.65 | 0.65 | No | Ca 3 |

This produced a gelation time of approximately 3 mintues for the mixture.
*grams of 0.4M TEMED solution

The Use of Stablizers and the Influence of Oxygen

| | | | | PEG | | | Stabiliser | | |
|---|---|---|---|---|---|---|---|---|---|
| | KSPA | AMP | | Acrylate | 0.1M | 0.1M | 0.02 g | Oxygen | Gelation |
| | (50%) | (50%) | H2O | ml | AA | OX | (0.1% w/w) | Exclusion | Time (min) |
| DPA1 | 3 g | 3 g | 1 g | 0.1 g | 0.65 g | 0.65 g | DPA | No | ca 5 |
| DPA2 | 3 g | 3 g | 1 g | 0.1 g | 0.65 g | 0.65 g | DPA | Yes | ca 3 |
| EDTPA1 | 3 g | 3 g | 1 g | 0.1 g | 0.65 g | 0.65 g | EDPTA | Yes | ca 3 |

### Abbreviations

sodium 2-acrylamido 2 methylpropane sulphonic acid (AMPS)
3-sulfopropyl ester acrylate (KSPA)
polyethylenglycol-400-diacrylate (IRR280)
potassium persulphate (KPS)
N,N,N′,N′-tetramethylethanediamine (TEMED)
oxone (OX)
ascorbic Acid (AA)
dipicolinic acid (DPA)
diethylene triamine penta(methylene phosphonic acid) (EDTPA)

1.-54. (canceled)

55. A method for repairing and/or preventing damage to an intervertebral disc or replacement lens comprising delivering an aqueous gel obtainable by polymerizing one or more olefinically unsaturated monomers in the presence of a cross-linking agent, said monomers comprising one or more phosphate, phosphonate, borate, sulphate and/or sulphonate groups to the intervertebral disc or lens.

56. The method of claim 55, wherein said one or more olefinically unsaturated monomers are selected from the group consisting of 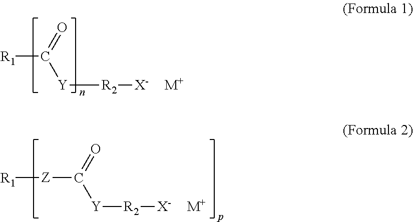
in which:
R₁=olefinically unsaturated moiety,
R₂=branched or unbranched, saturated or unsaturated, substituted or non-substituted alkyl, or substituted or unsubstituted aryl,
X=phosphate, phosphonate, borate, sulphate or a sulphonate group,
Y=—NH— or O,
Z=independently a single bond or —CH₂—,
M=a cation,
n=0 or 1, and
p=1 or 2.

57. The method of claim 55, wherein said aqueous gel further comprises an initiator.

58. The method of claim 55, wherein said monomers further comprise one or more physiologically compatible cations anionically bound to said monomer and selected from the group consisting of Na⁺, Ca²⁺, K⁺, NH4⁺, and combinations thereof.

59. The method of claim 55, wherein said monomer comprises one or more sulphonate groups.

60. The method of claim 56, wherein R₁ is selected from the group consisting of an ethenyl moiety and a propenyl moiety.

61. The method of claim 56, wherein R₂ contains from two to six carbon atoms.

62. The method of claim 56, wherein R₂ is selected from the group consisting of propyl, secondary butyl and tertiary butyl.

63. The method of claim 56, wherein X is sulphate or a sulphonate.

64. The method of claim 63, wherein said sulphonate is a C₁, C₂, C₃, C₄, C₅, C₆, C₇, C₈, C₉, C₁₀, C₁₁, C₁₂, C₁₃, C₁₄, C₁₅, C₁₆, C₁₇, or C₁₈ sulphonate.

65. The method of claim 55, wherein said monomers are anionic.

66. The method of claim 56, wherein said monomer is selected from sodium-2-(acrylamido)-2-methylpropane sulphonate and 3-sulphopropyl (meth)acrylate potassium salt.

67. The method of claim 55, wherein said aqueous gel further comprises a co-monomer.

68. The method of claim 67, wherein said co-monomer is selected from: one or more of an acrylic, methacrylic, acrylamido or vinyl compound capable of undergoing free radical polymerization, esters of unsaturated polyhydric alcohols (e.g. butenediol), vinyl cyclic compounds (e.g. styrene, vinyl furane, N-vinyl pyrrolidone), unsaturated acids (e.g. acrylic, methacrylic, propacrylic acid), unsaturated anhydrides (e.g. maleic, citraconic, itaconic), unsaturated nitriles (e.g. acrylonitrile, methacrylonitrile), unsaturated amines (e.g. acrylamide, dimethylaminoethyl methacryclate), vinyl halides (e.g. vinyl chloride, vinyl iodide, allyly chloride), unsaturated ketones (e.g. methyl vinyl ketone, ethyl vinyl ketone), unsaturated ethers (e.g. methyl vinyl ether, diallyl ether), unsaturated esters (e.g. hydroxylethyl methacrylate, hydroxypropyl acrylate), and unsaturated functional silanes, alkyl methacrylates (e.g. methyl methacrylate, ethyl methacrylate) and combinations thereof.

69. The method of claim 68, wherein said compound capable of undergoing free radical polymerization is selected from: 2-hydroxyethyl methacrylate (HEMA), glycerol methacrylate, polyethyleneglycol mono(meth)acrylate, methacrylic acid and acrylic acid, N,N-dimethyl acrylamide (DMA), 2-hydroxyethyl methacrylamide, 2-hydroxyethyl acrylamide, 2,2-di methoxy, 1-hydroxy acrylamide, hydroxymethyl diacetone acrylamide, N-acryloyl morpholine, N-(tris(hydroxymethyl)methyl)acrylamide, 2-hydroxylmethylacrylamide, an N-vinyl lactam, an N-vinyl amide, N-vinyl-N-methyl acetamide, N-vinyl-N-ethyl vinyl acetamide, N-vinyl-N-ethyl formamide, N-vinyl formamide and combinations thereof.

70. The method of claim 55, wherein said cross-linking agent is selected from methylene bisacrylamide, ethylene glycol dimethacrylate, polyethylglycol di(meth)acrylate, tetraethylene glycol dimethacrylate, diallyl tartramide, pentaerithratol diacrylate, divinyl benzene and polyethylene glycol diacrylate.

71. The method of claim 55, wherein said polymer comprises a water-soluble interpenetrant selected from acrylamide and a substituted vinyl amide.

72. The method of claim 57, wherein said initiator is a two-part initiator comprising a reducing agent and an oxidising agent.

73. The method of claim 72, wherein the reducing agent comprises ascorbic acid, N,N-dimethyl toluidine, hydroxymethane sulfinic acid and salts thereof, and/or sodium metabisulfite.

74. The method of claim 72, wherein said oxidising agent comprises potassium persulphate, oxypersulphate, peroxide, benzoyl peroxide, t-butyl hydroperoxide, di(isopropyl) peroxydicarbonate, t-butyl peroctoate, bis(4-t-butylcyclohexyl) peroxydicarbonate and isopropyl peroctoate.

75. The method of claim 55, wherein said aqueous gel further comprises a radiopaque marker.

76. The method of claim 72, wherein said monomers are delivered to a target site with a multiple-barrel syringe, a first barrel of the syringe containing a first part of the two-part initiator and a second barrel of the syringe containing a second part of the two part initiator such that on expulsion from the syringe, the monomers and the first and second parts of the two-part initiator come into contact causing the monomers to polymerize at the target site.

77. The method of claim 55, wherein said aqueous gel is sterilized prior to use.

78. A kit comprising a syringe and an aqueous gel aqueous gel obtainable by polymerizing one or more olefinically unsaturated monomers in the presence of a cross-linking agent, said monomers comprising one or more phosphate, phosphonate, borate, sulphate and/or sulphonate groups to the intervertebral disc or lens.

79. A multiple barrel syringe containing one or more olefinically unsaturated monomers of one of the formulae: 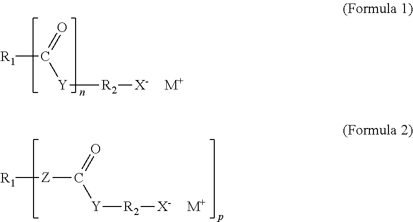
or in which:
R₁=olefinically unsaturated moiety,
R₂=branched or unbranched, saturated or unsaturated, substituted or non-substituted alkyl, or substituted or unsubstituted aryl,
X=phosphate, phosphonate, borate, sulphate or a sulphonate group,
Y−—NH— or O,
Z=independently a single bond or —CH₂—,
M=a cation,
n=0 or 1; and
p=1 or 2, and
(a) a first barrel of the syringe contains a first part of a two-part initiator and
(b) second barrel of the syringe contains a second part of a two-part initiator
such that when the contents of the syringe are released from the syringe, the monomers, the first part of the two-part initiator and the second part of the two-part initiator contact each other causing polymerization of the monomers to form an aqueous polymeric gel.
